(19) United States
(12) Patent Application Publication
(10) Pub. No.: US 2025/0145618 A1
(43) Pub. Date: May 8, 2025

(54) Crystalline Forms of (R)-1-(1-Acryloylpiperidin-3-YL)-4-Amino-3-(4-Phenoxyphenyl)-1H-Imidazo[4,5-C]Pyridin-2(3H)-One and salts Thereof

(71) Applicants: Principia Biopharma Inc., Bridgewater, NJ (US); GENZYME CORPORATION, Cambridge, MA (US)

(72) Inventors: Tim Owens, Belmont, CA (US); Philippe Eric Ochsenbein, Montpellier Cedex 04 (FR); Krista Diaz

(73) Assignee: Principia Biopharma Inc., Bridgewater, NJ (US)

(21) Appl. No.: 18/722,486

(22) PCT Filed: Dec. 20, 2022
(86) PCT No.: PCT/US2022/053479

§ 371 Date: Jun. 20, 2024

Related U.S. Application Data
(60) Provisional application No. 63/432,169, filed on Dec. 13, 2022.
(60) Provisional application No. 63/292,124, filed on Dec. 21, 2021.

(51) Int. Cl. C07D 471/04

(52) U.S. Cl. CPC C07D 471/04

(57) ABSTRACT

The present disclosure relates to crystalline forms of (R)-1-(1-acryloylpiperidin-3-yl)-4-amino-3-(4-phenoxyphenyl)-1H-imidazo[4,5-c]pyridin-2(3H)-one free base and HCl salt thereof.

### FIELD

Disclosed herein are crystalline forms of (R)-1-(1-acryloylpiperidin-3-yl)-4-amino-3-(4-phenoxyphenyl)-1H-imidazo[4,5-c]pyridin-2(3H)-one free base (also referred to herein as Compound (1)), having the structure:

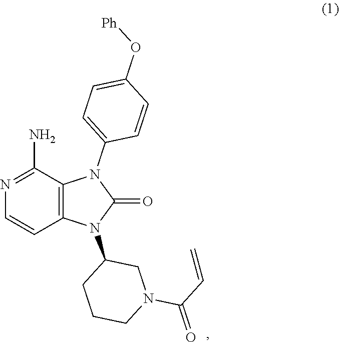 [SMILES: C=CC(=O)N1CCC[C@@H](n2c(=O)n(-c3ccc(Oc4ccccc4)cc3)c3c(N)nccc32)C1]

as well as derivatives and forms thereof. Compound (1) and its salts and solid state forms thereof are potent Bruton's Tyrosine Kinase (“BTK”) inhibitors and thus can be useful in the treatment of diseases or disorders resulting from an excess of BTK signaling.

### BACKGROUND

One factor in assessing the suitability of a compound as a therapeutic agent is whether the compound may be synthesized in a manner that is amenable to large scale manufacturing and isolation, with minimal product waste and impurities. This factor is frequently considered when reviewing the suitability of a bench-scale process for making the larger quantities needed for commercial production. For example, Compound (1) and a method for preparing it is disclosed in Example 3 of U.S. Pat. No. 9,688,676, herewith:

Into a 100-mL round-bottom flask, was placed (R)-4-amino-3-(4-phenoxyphenyl)-1-(piperidin-3-yl)-1H-imidazo[4,5-c]pyridin-2(3H)-one (150 mg, 0.37 mmol, 1.00 equiv), DCM-CH₃OH (6 mL), TEA (113 mg, 1.12 mmol, 3.00 equiv). This was followed by the addition of prop-2-enoyl chloride (40.1 mg, 0.44 mmol, 1.20 equiv) dropwise with stirring at 0° C. in 5 min. The resulting solution was stirred for 2 h at 0° C. The resulting mixture was concentrated under vacuum. The residue was applied onto a silica gel column with dichloromethane/methanol (30:1). The crude product (100 mg) was purified by Prep-HPLC with the following conditions (Column, XBridge Prep C18 OBD Column, 5 μm, 19*150 mm; mobile phase, water with 0.05% TFA and ACN (25.0% ACN up to 45.0% in 8 min). As noted above, this synthesis provides 100 mg of crude Compound (1) that must be purified by column chromatography, resulting in 54.5 mg of purified Compound (1).

Another, desirable aspect to be achieved is that the compound as a therapeutic agent can be administered in a form that is easily absorbed by the body and also shelf-stable. The pharmaceutically active substance used to prepare the treatment should be as pure as possible and its stability on long-term storage should be guaranteed under various environmental conditions. These properties are useful to prevent the appearance of unintended degradation products in pharmaceutical compositions, which degradation products may be potentially toxic or result simply in reducing the potency of the composition.

A primary concern for the large-scale manufacture of pharmaceutical compounds is that the active substance should have a stable crystalline morphology to ensure consistent processing parameters and pharmaceutical quality. If an unstable crystalline form is used, crystal morphology may change during manufacture and/or storage, resulting in quality control problems and formulation irregularities. Such a change may affect the reproducibility of the manufacturing process and thus lead to final formulations which do not meet the high quality and stringent requirements imposed on formulations of pharmaceutical compositions. In this regard, it should be generally borne in mind that any change to the solid state of a pharmaceutical composition which can improve its physical and chemical stability gives a significant advantage over less stable forms of the same drug.

When a compound crystallizes from a solution or slurry, it may crystallize with different spatial lattice arrangements, a property referred to as “polymorphism.” Each of the crystal forms is a “polymorph.” Although polymorphs of a given substance have the same chemical composition, they may differ from each other with respect to one or more physical properties, such as solubility, dissociation, true density, dissolution, melting point, crystal shape, compaction behavior, flow properties, and/or solid state stability.

### BRIEF SUMMARY

In accordance with the description, the present disclosure relates to a substantially crystalline compound of Formula (1).

In one embodiment, the substantially crystalline compound of Formula (1) is a free base.

In another embodiment, the substantially crystalline compound of Formula (1) is a compound of Formula (1)·HCl.

Additional objects and advantages will be set forth in part in the description which follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIG. 1 shows an XRPD pattern of Compound (1) crystalline free base Form 1 obtained using CuKα radiation.

FIG. 2 shows an XRPD pattern of Compound (1) crystalline free base Form 2 obtained using CuKα radiation.

FIG. 3A shows an ORTEP representation of the molecular structure of Compound (1) crystalline free base Form 1.

FIG. 3B shows an ORTEP representation of the molecular structure of Compound (1) crystalline free base Form 1 down the short axis: representation of the molecular packing focusing on the hydrogen bonding network (dotted lines).

FIG. 4 shows a simulated powder diffraction pattern from the single crystal structure of Compound (1) crystalline free base Form 1.

FIG. 5 shows an XRPD pattern of Compound (1)·HCl crystalline Form 1 obtained using CuKα radiation.

FIG. 6A shows an ORTEP representation of the molecular structure of Compound (1)·HCl crystalline Form 1.

FIG. 6B shows an ORTEP representation of the molecular structure of Compound (1)·HCl crystalline Form 1 down the b axis: representation of the molecular packing.

FIG. 7 shows a simulated powder diffraction pattern from the single crystal structure of Compound (1)·HCl crystalline Form 1.

FIG. 8 shows an XRPD pattern of Compound (1)·HCl crystalline Form 2 obtained using CuKα radiation.

FIG. 9A shows an ORTEP representation of the molecular structure of Compound (1)·HCl crystalline Form 2.

FIG. 9B shows an ORTEP representation of the molecular structure of Compound (1)·HCl crystalline Form 2: representation of the molecular packing.

FIG. 10 shows a simulated powder diffraction pattern from the single crystal structure of Compound (1)·HCl crystalline Form 2.

### DETAILED DESCRIPTION

Reference will now be made in detail to certain embodiments, examples of which are illustrated in the accompanying drawings. While the disclosure provides illustrated embodiments, it will be understood that they are not intended to limit the invention to those embodiments. On the contrary, the invention is intended to cover all alternatives, modifications, and equivalents, which may be included within the disclosure as defined by the appended claims.

Any section headings used herein are for organizational purposes only and are not to be construed as limiting the desired subject matter in any way. In the event that any literature incorporated by reference contradicts any term defined in this specification, this specification controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

### I. Definitions

Unless otherwise stated, the following terms used in the specification and claims are defined for the purposes of this disclosure and have the following meanings.

As used herein, “the BTK inhibitor,” “the BTK inhibitor compound,” “the compound of Formula (1),” “Compound (1),” and “the compound,” refers to (R)-1-(1-acryloylpiperidin-3-yl)-4-amino-3-(4-phenoxyphenyl)-1H-imidazo[4,5-c]pyridin-2(3H)-one, having the following structure:

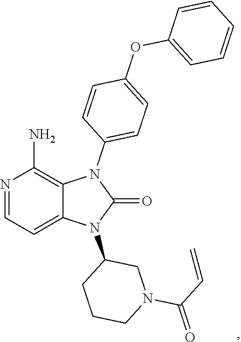 [SMILES: C=CC(=O)N1CCC[C@@H](n2c(=O)n(-c3ccc(Oc4ccccc4)cc3)c3c(N)nccc32)C1]

which is also known as “tolebrutinib,” and 4-amino-3-(4-phenoxyphenyl)-1-[(3R)-1-(prop-2-enoyl)piperidin-3-yl]-1,3-dihydro-2H-imidazo[4,5-c]pyridin-2-one having the following structure:

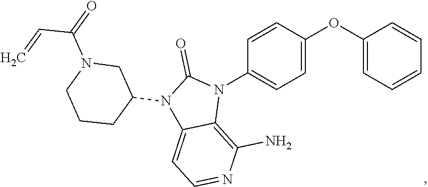

and/or a pharmaceutically acceptable salt thereof.

The present disclosure relates to a substantially crystalline compound of Formula (1).

In some embodiments, the substantially crystalline compound of Formula (1) is at least 50% crystalline, such as at least 60%, at least 70%, at least 80%, at least 90%, at least 95%, at least 96%, at least 97%, at least 98%, at least 99%, or 100% crystalline.

As used herein, the term “crystalline” or “crystalline solid form,” refers to a solid form which is substantially free of any amorphous solid-state form.

In some embodiments, “substantially free” means less than about 10% w/w, less than about 9% w/w, less than about 8% w/w, less than about 7% w/w, less than about 6% w/w, less than about 5% w/w, less than about 4% w/w, less than about 3% w/w, less than about 2.5% w/w, less than about 2% w/w, less than about 1.5% w/w, less than about 1% w/w, less than about 0.75% w/w, less than about 0.50% w/w, less than about 0.25% w/w, less than about 0.10% w/w, or less than about 0.05% w/w of other crystalline forms of the compound and the amorphous compound. In some embodiments, “substantially free” means an undetectable amount of other crystalline forms of the compound and the amorphous compound.

As used herein, the term “substantially pure” or “substantially crystalline” means that the crystalline form contains at least 90 percent, for example at least 95 percent, such as at least 97 percent, and even at least 99 percent by weight of the indicated crystalline form compared to the total weight of the compound of all forms.

Alternatively, it will be understood that “substantially pure” or “substantially crystalline” means that the crystalline form contains less than 10 percent, for example less than 5 percent, such as less than 3 percent, and even less than 1 percent by weight of impurities, including other polymorphic, solvated or amorphous forms compared to the total weight of the compound of all forms.

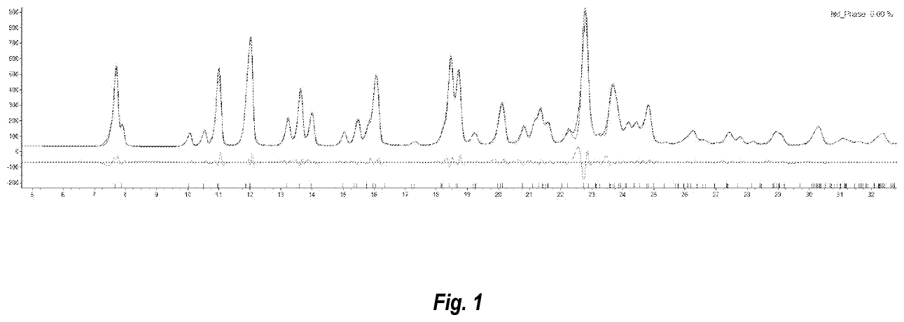

In some embodiments, the substantially crystalline compound of Formula (1) is Form 1. In at least one embodiment, the substantially crystalline compound of Formula (1) Form 1 is characterized by an XRPD pattern substantially the same as FIG. 1. In at least one embodiment, the substantially crystalline compound of Formula (1) Form 1 is characterized by an XRPD pattern comprising one or more peaks chosen from peaks at about 7.66° 2θ, 7.86° 2θ, 10.03° 2θ, 10.51° 2θ, 10.97° 2θ, 11.99° 2θ, 13.19° 2θ, 13.59° 2θ and 13.96° 2θ.

In some embodiments, the crystalline solid form characterized as crystalline Form 1 is at least 50% crystalline form, such as at least 60%, at least 70%, at least 80%, at least 90%, at least 95%, at least 96%, at least 97%, at least 98%, at least 99%, or 100% crystalline.

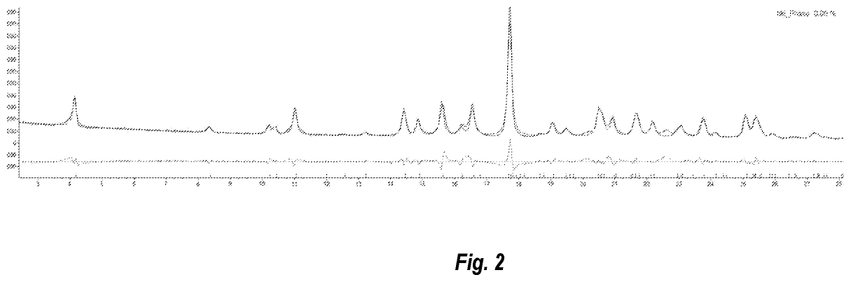

In some embodiments, the substantially crystalline compound of Formula (1) is Form 2. In at least one embodiment, the substantially crystalline compound of Formula (1) Form 2 is characterized by an XRPD pattern substantially the same as FIG. 2. In at least one embodiment, the substantially crystalline compound of Formula (1) Form 2 is characterized by an XRPD pattern comprising one or more peaks chosen from peaks at about 4.15° 2θ, 10.22° 2θ, 10.41° 2θ, 11.03° 2θ, 14.41° 2θ, 14.85° 2θ, 15.63° 2θ, 16.55° 2θ and 17.73° 2θ.

In some embodiments, the crystalline solid form characterized as crystalline Form 2 is at least 50% crystalline form, such as at least 60%, at least 70%, at least 80%, at least 90%, at least 95%, at least 96%, at least 97%, at least 98%, at least 99%, or 100% crystalline.

The present disclosure also relates to a substantially crystalline form of a compound of Formula (1)·HCl.

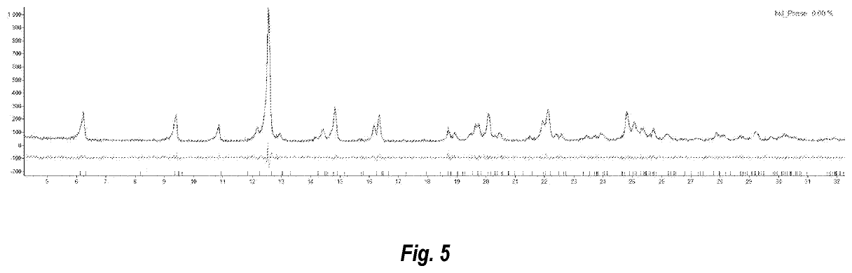

In some embodiments, the substantially crystalline compound of the compound of Formula (1)·HCl is Form 1. In at least one embodiment, the substantially crystalline compound of Formula (1)·HCl Form 1 is characterized by an XRPD pattern substantially the same as FIG. 5. In at least one embodiment, the substantially crystalline compound of Formula (1)·HCl Form 1 is characterized by an XRPD pattern comprising one or more peaks chosen from peaks at about 6.309° 2θ, 9.480° 2θ, 10.933° 2θ, 12.261° 20, 12.647° 2θ, 14.482° 2θ, 14.918° 2θ, 16.253° 2θ and 16.425° 2θ.

In some embodiments, the crystalline solid form characterized as crystalline Formula (1)·HCl Form 1 is at least 50% crystalline form, such as at least 60%, at least 70%, at least 80%, at least 90%, at least 95%, at least 96%, at least 97%, at least 98%, at least 99%, or 100% crystalline.

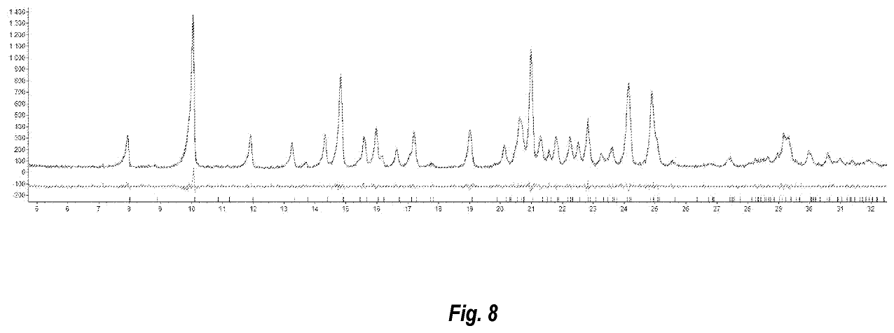

In some embodiments, the substantially crystalline compound of Formula (1)·HCl is Form 2. In at least one embodiment, the substantially crystalline compound of Formula (1)·HCl Form 2 is characterized by an XRPD pattern substantially the same as FIG. 8. In at least one embodiment, the substantially crystalline compound of Formula (1)·HCl Form 2 is characterized by an XRPD pattern comprising one or more peaks chosen from peaks at about 8.00° 2θ, 10.11° 2θ, 11.98° 2θ, 13.33° 2θ, 14.40° 2θ, 14.92° 2θ, 15.66° 2θ, 16.05° 2θ, 16.72° 2θ, and 17.28° 2θ.

In some embodiments, the crystalline solid form characterized as crystalline Formula (1)·HCl Form 2 is at least 50% crystalline form, such as at least 60%, at least 70%, at least 80%, at least 90%, at least 95%, at least 96%, at least 97%, at least 98%, at least 99%, or 100% crystalline.

In some embodiments, the substantially crystalline compound of Formula (1) is at least 85% crystalline. In some embodiments, the substantially crystalline compound of Formula (1) is at least 90% crystalline. In some embodiments, the substantially crystalline compound of Formula (1) is at least 95% crystalline. In some embodiments, the substantially crystalline compound of Formula (1) is at least 97% crystalline. In some embodiments, the substantially crystalline compound of Formula (1) is at least 99% crystalline.

The following abbreviations may be relevant for this application.

### Abbreviations

| | |
|---|---|
| ACN | acetonitrile |
| AcOEt | ethyl acetate |
| DBU | 1,8-diazabicyclo[5.4.0]undec-7-ene |
| DCM | dichloromethane |
| DIEA | diethylamine |
| DIPEA | diisopropylethylamine |
| DMF/DMA | dimethylformamide/dimethylacetamide |
| DSC | Differential Scanning Calorimetry |
| EtOH | ethanol |
| hr | hour(s) |
| iPrOAc | isopropylacetate |
| min | minute(s) |
| MTBE | methyl tert-butyl ether |
| T3P | propylphosphonic anhydride |
| TG | thermogravimetry |
| vol. | volume |

### II. EXAMPLES

### Example 1

### Characterization of Compound (1)

Compound (1), made according to the method disclosed in the above-mentioned U.S. Pat. No. 9,688,676, underwent crystallization attempts, resulting in two forms as determined by XRPD analysis.

### 1.1 Compound (1) Crystalline Form 1

Ethyl acetate (AcOEt) was added to Compound (1) and heated to 50° C. The heating device was turned off and the sample was allowed to cool to ambient temperature. The solids on the bottom of the vial were scraped and slurried with AcOEt at RT for 2 days. Additional AcOEt was added and the slurry sat at RT for 3 days. Additional AcOEt was added and then the solution underwent vacuum filtration.

Compound 1·Form 1 underwent XRPD analysis performed on a Bruker D2-Phaser diffractometer following these parameters:
Source CuKal, 1=1.5406Å.
Generator: 30 kV-10 mA.
Detector: Lynxeye SSD160 (1D mode)
Powder specimen holder
Rotating sample holder: 30 rpm
Angle range: 2° to 40° in 2-theta Bragg.
Step size: 0.03°
Step time: 0.5s by Step
PSD opening: 4.8°
Detector slit: 8 mm
X-Ray generator slit: 0.6 mm
Sample preparation: gently grinding
Space Group: P21
The unit cell parameters are given below:
   a(Å)=8.9182 b(Å)=11.7707 c(Å)=11.9324 α(°)=97.197 β(°)=107.211 γ(°)=96.440

FIG. 1 shows an XRPD pattern of Compound (1) Form 1 obtained using Cu Kα radiation (wavelength: λ(Cu)=1.54178 Å).

Peaks identified in FIG. 1 include those set forth in Table 1:

TABLE 1

| Pos. [°2θ] | d-spacing [Å] | Height [cts] |
|---|---|---|
| 7.66 | 11.532 | 554.5 |
| 7.86 | 11.238 | 173.7 |
| 10.03 | 8.818 | 119.1 |
| 10.51 | 8.413 | 139.2 |
| 10.97 | 8.059 | 543.0 |
| 11.99 | 7.372 | 736.8 |
| 13.19 | 6.705 | 211.8 |
| 13.59 | 6.511 | 397.8 |
| 13.96 | 6.338 | 248.6 |

### Example 1.2 Compound (1) Crystalline Form 2

Isopropyl acetate (iPrOAc) was added to Compound 1 to form a slurry, which sat at RT for 3 days, then as a cold slurry for 4 days. More iPrOAc was added, followed by RT slurry for 1 day. Additional iPrOAc was added and then the solution underwent vacuum filtration.

Compound 1·Form 2 underwent XRPD analysis performed on a Bruker D2-Phaser diffractometer following these parameters:
Source CuKal, 1=1.5406Å.
Generator: 30 kV-10 mA.
Detector: Lynxeye SSD160 (1D mode)
Powder specimen holder
Rotating sample holder: 30 rpm
Angle range: 2° to 40° in 2-theta Bragg.
Step size: 0.03°
Step time: 0.5s by Step
PSD opening: 4.8°
Detector slit: 8 mm
X-Ray generator slit: 0.6 mm
Sample preparation: gently grinding
Space Group: P21
The unit cell parameters are given below:
   a(Å)=8.6381 b(Å)=42.2015 c(Å)=6.1873 α(°)=90.000 β(°)=90.433 γ(°)=90.000

FIG. 2 shows an XRPD pattern of Compound (1) ·Form 2 obtained using Cu Kα radiation (wavelength: 1(Cu)=1.54178 Å). Peaks identified in FIG. 2 include those set forth in Table 2:

TABLE 2

| Pos. [°2θ] | d-spacing [Å] | Height [cts] |
|---|---|---|
| 4.15 | 21.298 | 384.5 |
| 10.22 | 8.648 | 158.3 |
| 10.41 | 8.488 | 144.7 |
| 11.03 | 8.015 | 292.4 |
| 14.41 | 6.143 | 260.9 |
| 14.85 | 5.960 | 192.3 |
| 15.63 | 5.666 | 325.4 |
| 16.55 | 5.353 | 300.4 |
| 17.73 | 5.000 | 1174.9 |

### Example 2

### Single Crystal Data for Compound (1) Form 1

A single crystal from a batch made as described in Example 1.1 was selected by observation under a binocular microscope was mounted on the goniometric head of a Bruker APEX2 Instrument diffractometer (Bruker AXS (2011).APEX2 suite V2011.2-0 Madison, Wisconsin, U.S.A.). Intensities were collected at low temperature (T=112 K), with the use of a microfocus ImuS Cu Kα radiation wavelength of (λ=1.54178 Å). Systematic investigation of the diffraction nodes indicates that the crystal belongs to the triclinic system, with a primitive Bravais lattice. The unit cell parameters are given below:
a(Å)=8.81 b(Å)=11.58 c(Å)=11.77 α(°)=97.75 β(°)=107.23 γ(°)=94.97

In view of the number of atoms in the Compound (1) Form 1 molecule and of the unit cell volume, it was concluded that this unit cell must contain two molecules having the formula C₂₆H₂₅N₅O₃ which is equivalent to a calculated density of 1.342. The number of reflections collected was 27267, of which 7140 were unique.

Based on the statistical distribution of the intensities, a non-centrosymmetric structure is deduced.

The structure was solved by direct methods using the XT dual-space module of SHELX; and was refined on F² by full least squares methods with SHELXTL, as set forth in Sheldrick, G. M. “A short history of SHELX,” Acta Crystallogr. Sect. A (2008) A64, 112-122. All non-hydrogen atoms were refined with anisotropic displacement parameters; a riding model was used for hydrogen atoms. Final agreement values are R1=0.0267 (observed reflections) and wR2=0.0722 (all data) for 7140 reflections and 625 parameters, with a goodness of fit of 1.242.

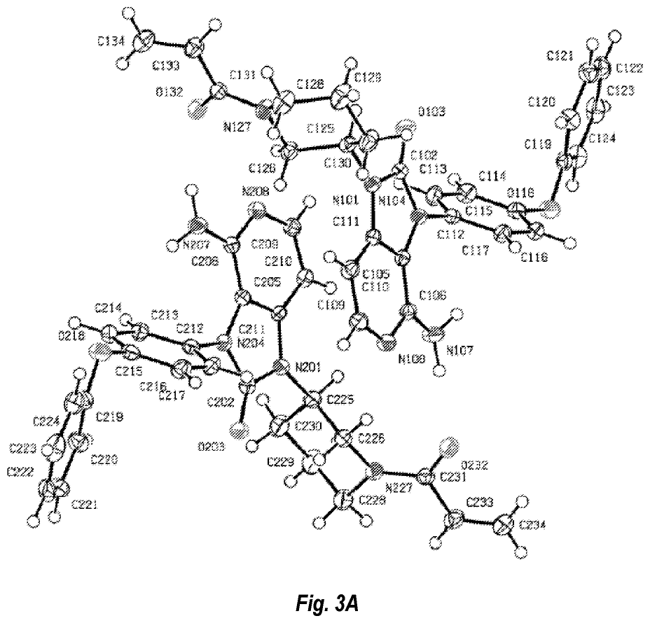

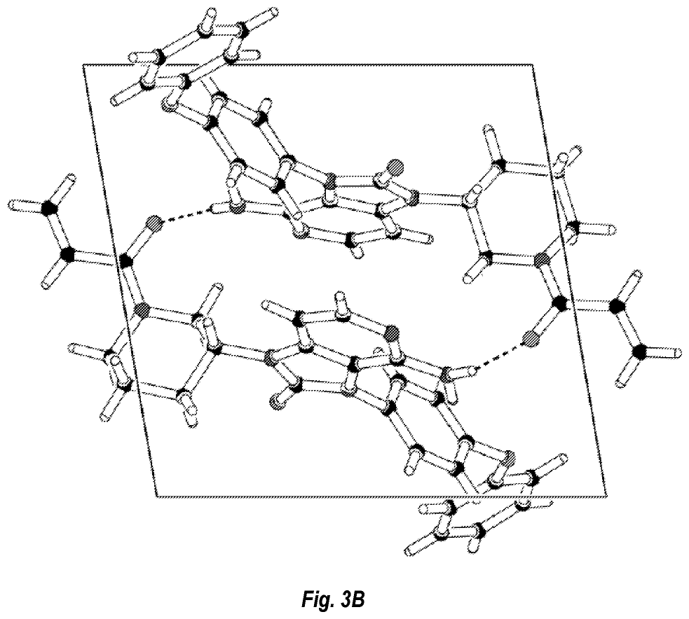

Compound (1) crystallizes in the space group P 1, the asymmetric unit of the crystal is made up of 2 molecules of Compound (1) Form 1, thus 2 formulae are present in the unit cell. See FIGS. 3A and 3B. No additional molecule like organic solvent or water was found. Examination of the molecular structure confirmed that all bond angles and lengths stand in the standard range values. No disorder seemed to be present in the crystal.

Crystal data, X-rays experimental parameters and structure refinements are given in Table 3.

TABLE 3
Crystal Data and Structure Refinements of Compound
(1) Form 1 by Single Crystal X-Ray Diffraction

| Identification | Compound 1 Form 1 |
|---|---|
| Chemical formula | C₂₆ H₂₅ N₅ O₃ |
| Molecular weight | 455.51 |
| Temperature | 112(2) |
| Wavelength | 1.54178 |
| Crystal system; space group | triclinic; P 1 |
| | a = 8.81320(10) Å; α = 97.7520(10) ° |
| Unit cell dimensions | b = 11.5833(2) Å; β = 107.2300(10) ° |
| | c = 11.7701(2) Å; γ = 94.9650(10) ° |
| Volume | 1126.95(3) Å³ |
| Z, Calculated density | 2, 1.342 Mg/m³ |
| Absorption coefficient | 0.733 1/mm |
| F(000) | 480 |
| Theta range for data collection | 3.886° to 63.740° |
| Limiting indices | −10 <= h <= 10; −13 <= k <= |
| | 13; −13 <= l <= 13 |
| Reflexion collected/unique | 27267/7140 [R(int) = 0.0327] |
| Completeness to theta max | 99.1% |
| Refinement method | Full-matrix least-square on F² |
| Data/restraints/parameters | 7140/463/625 |
| Goodness of fit on F² | 1.242 |
| Final R indices [I > 2sigma(I)] | R1 = 0.0267; wR2 = 0.0711 |
| Final R indices [all data] | R1 = 0.0288; wR2 = 0.0722 |
| Absolute structure parameter | 0.05(7) |
| Largest diff peak and hole | 0.134 and −0.144 e/Å3 |

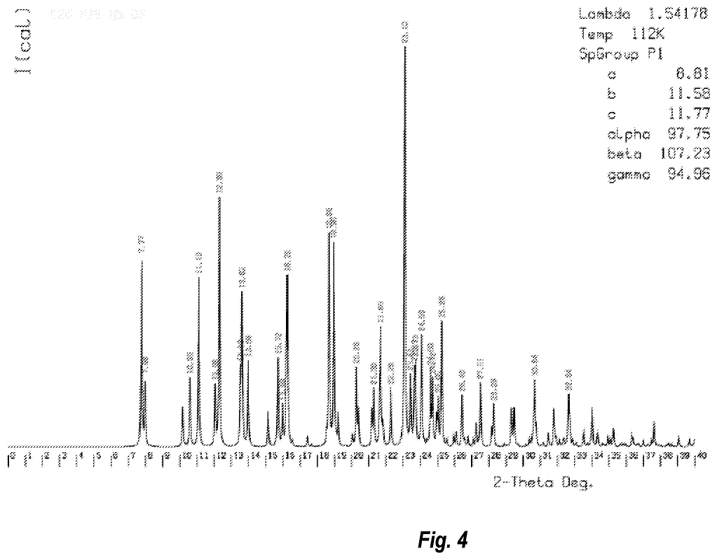

A simulated diffraction pattern (FIG. 4) was produced from the experimentally determined crystalline structure. An experimental powder diffraction pattern can be compared to this theoretical pattern to demonstrate the nature of the crystalline structure. Minor differences (if any) can be explained by asymmetric crystal morphology, particle size, or preferential orientations in the powder.

### Example 3

### Synthesis of and Characterization of Compound (1)·HCl

Overview. Compound (1). HCl was prepared as shown in the following scheme. The batch size of these reactions was typically 14 to 60 kg, and can be carried out at a scale of up to about 100 kg.

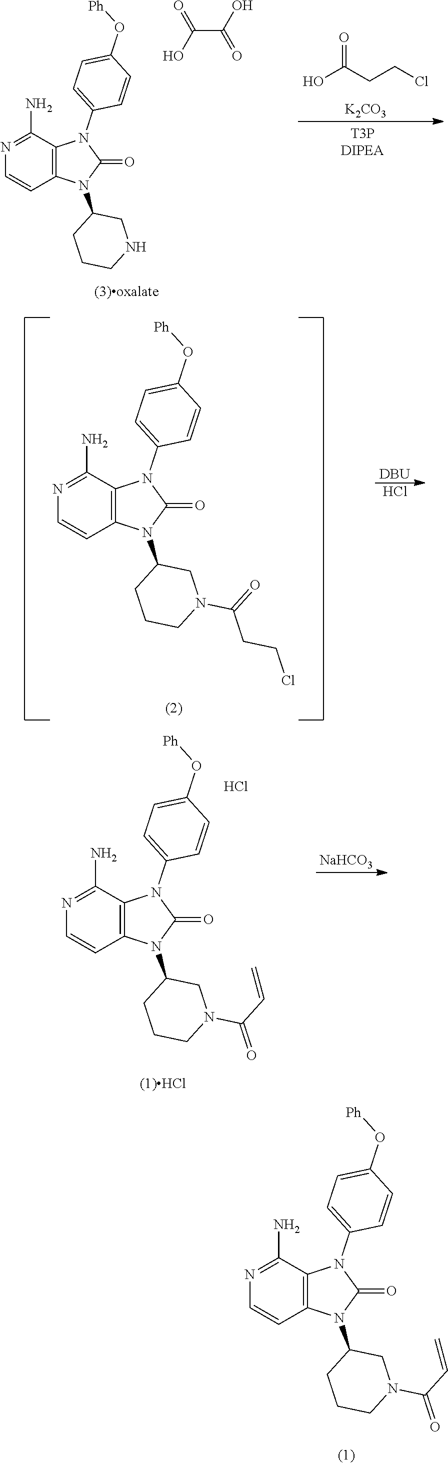

### Detailed Synthesis

### 2.1 Preparation of Compound (1)·HCl.

Purified water (7.5 vol.) and K₂CO₃ (at least 3.0 eq.) was added to Compound (3)·oxalate (hydrate; corresponds to 1 eq. Compound (3)) in DCM (12 vol.) at 20° C., and the reaction mixture was stirred for at least 2 hr. The reaction mixture was then allowed to settle and separate. The organic layers were collected and washed 1-2 times with water (7.5 vol.) to afford Compound (3) in DCM solution. The solution was concentrated to 12 vol. and mixed with DIPEA (4 eq.) at 20° C. Next, a solution of 3-chloropropanoic acid (1.05 eq.) in DCM (2.3 vol.) and T3P (50% DCM solution, 1 eq.) was added at 20° C. Compound 2 was formed in situ. Next, DBU (4 eq.) was added to the reaction mixture at 30° C. over at least 30 min. and the resulting mixture was kept at 30° C. for at least 2 hr. The organic layer was washed 3-5 times with HCl (1 N, 10 vol.) at 20° C. Next, the organic layer was concentrated to 2.73 vol. and the temperature was adjusted to 35° C. Compound 1·HCl seeds (0.1 kg/kg) were added to the organic layer at 35° C. and the temperature was maintained for at least 1 hr. Ethyl acetate (2 vol.) was then added and a temperature of 35° C. was maintained for at least 1 hr. Next, the reaction mixture was cooled to 10° C. and ACN (1.07 vol.) was added. The mixture was cooled to 0° C. A filter-dryer was charged with the resulting suspension and re-slurried with DCM (0.72 vol.)/AcOEt (0.63 vol.)/ACN (0.45 vol.) at 0° C. The solid was filtered, washed twice with AcOEt (1.8 vol.), and twice with ACN (1.8 vol.). The resulting Compound 1·HCl was dried at a maximum temperature of 50° C. The following elemental analysis was performed by Galbraith Laboratories: Carbon, Hydrogen, and Nitrogen Determination using the PerkinElmer2400 Series II CHNS/O Analyzer and determination of Total Halogens or Total Halides by Potentiometric Titration.

| | Element | Relative weight | Calculated |
|---|---|---|---|
| EA | C | 62.94% | 63.48% |
| | H | 5.48% | 5.33% |
| | N | 13.92% | 14.24% |
| | C1 | 6.49% | 7.21% |

### 3.2 Crystal Data for Compound (1)·HCl Form 1

Ethyl acetate (AcOEt) was added to Compound (1) HCl and heated to 50° C. The heating device was turned off and the sample was allowed to cool to ambient temperature. The solids on the bottom of the vial were scraped and slurried with AcOEt at RT for 2 days. Additional AcOEt was added and the slurry sat at RT for 3 days. Additional AcOEt was added and then the solution underwent vacuum filtration.

Compound 1·HCl underwent XRPD analysis performed on a Bruker D2-Phaser diffractometer following those parameters:
Source CuKal, 1=1.5406Å.
Generator: 30 kV-10 mA.
Detector: Lynxeye SSD160 (1D mode)
Powder specimen holder
Rotating sample holder: 30 rpm
Angle range: 2° to 40° in 2-theta Bragg.
Step size: 0.03°
Step time: 0.5s by Step
PSD opening: 4.8°
Detector slit: 8 mm
X-Ray generator slit: 0.6 mm
Sample preparation: gently grinding
Space Group: P21
The unit cell parameters are given below:
   a(Å)=14.1097 b(Å)=12.2212 c(Å)=14.5523 α(°)=90.00 β(°)=97.653 γ(°)=90.00

FIG. 5 shows an XRPD pattern of Compound (1)·HCl Form 1 obtained using Cu Kα radiation (wavelength: λ(Cu)=1.54178 Å).

Peaks identified in FIG. 4 include those set forth in Table 4:

TABLE 4

| Pos. [°2θ] | d-spacing [Å] | Height [cts] |
|---|---|---|
| 6.31 | 13.984 | 261.3 |
| 9.48 | 9.324 | 228.7 |
| 10.93 | 8.085 | 158.3 |
| 12.26 | 7.211 | 131.4 |
| 12.65 | 6.992 | 1056.0 |
| 14.48 | 6.110 | 124.1 |
| 14.92 | 5.934 | 291.0 |
| 16.25 | 5.449 | 155.6 |
| 16.43 | 5.391 | 231.0 |

### 3.3 Single Crystal Data and Structure Refinements of Compound (1)·HCl Form 1

A single crystal of Compound 1·HCl Form 1 (grown in a mixture of ACN and DCM) was selected by observation under a binocular microscope was mounted on the goniometric head of a Bruker APEX DUO Instrument equipped with a micro focused X-ray source. (Bruker AXS (2015).APEX3 suite V2014.2-0 Madison, Wisconsin, U.S.A). Intensities were collected with the diffractometer at low temperature (T=100 K), with the use of a graphite monochromated Cu Kα radiation wavelength (λ=1.54178 Å). Systematic investigation of the diffraction nodes indicates that the crystal belongs to the monoclinic system, with a primitive Bravais lattice. The unit cell parameters are given below:

a(Å)=13.62 b(Å)=12.06 c(Å)=14.74 α(°)=90.00 β(°)=97.06 γ(°)=90.00

In view of the number of atoms in the Compound (1)·HCl Form 1 molecule and of the unit cell volume, it was concluded that this unit cell must contain four molecules having the formula C₂₆H₂₆ClN₅O₃, which is equivalent to a calculated density of 1.359. The number of reflections collected was 35059, of which 8540 were unique.

Determination of the space group was achieved unequivocally due to the presence of a unique systematic extinction along the monoclinic axis.

The structure was solved by direct methods using the XT dual-space module of SHELX; and was refined on F2 by full least squares methods with SHELXTL, as set forth in Sheldrick, G. M. “A short history of SHELX,” Acta Crystallogr. Sect. A (2008) A64, 112-122. All non-hydrogen atoms were refined with anisotropic displacement parameters; a riding model was used for hydrogen atoms. Final agreement values are R1=0.0352 (observed reflections) and wR2=0.1131 (all data) for 8540 reflections and 631 parameters, with a goodness of fit of 0.917.

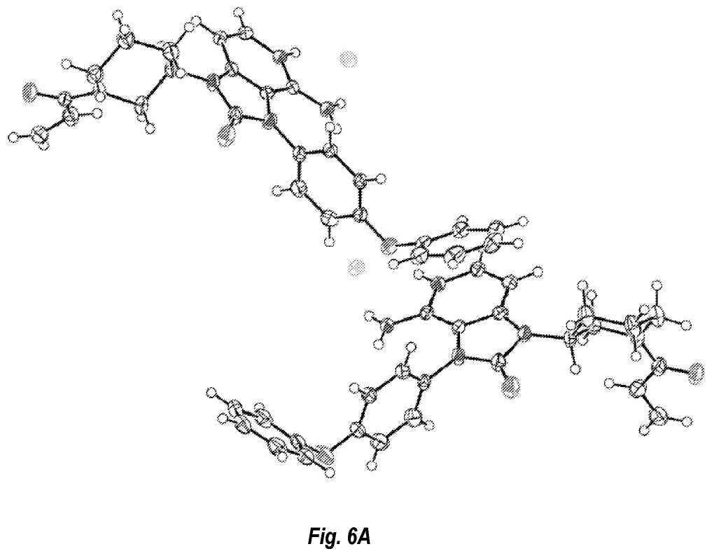

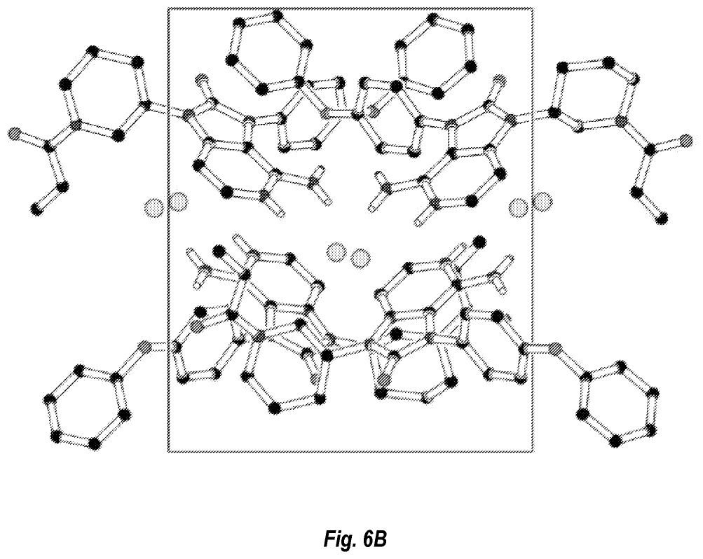

The compound crystallizes in the space group P 21, the asymmetric unit of the crystal is made up of two molecules of Compound 1 associated to their respective counter ion, and thus four formulae are present in the unit cell. See FIGS. 6A and 6B. The asymmetric cell therefore contains: 2[C₂₆H₂₆N₅O₃, Cl]. No additional molecule like organic solvent or water is found. Examination of the molecular structure confirms that all bond angles and lengths stand in the standard range values. There is no atomic disorder in the crystal. The salt bridge is established by the chlorine atom with the amino-imidazolopyridine nitrogen atom. Other non-covalent interactions are also present in the structure.

Crystal data, X-rays experimental parameters and structure refinements are given in Table 5.

TABLE 5
Crystal Data and structure refinements of Compound
1•HCl Form 1 by Single Crystal X-ray Diffraction

| Identification | Compound 1•HCl Form 1 |
|---|---|
| Chemical formula | C₂₆ H₂₅ N₅ O₃ |
| Molecular weight | 491.97 |
| Temperature | 100(2) |
| Wavelength | 1.54178 |
| Crystal system; space group | monoclinic; P 21 |
| | a = 13.6222(5) Å; α = 90° |
| Unit cell dimensions | b = 12.0623(5) Å; β = 97.059(2) ° |
| | c = 14.7427(6) Å; γ = 90° |
| Volume | 2404.09(17) Å³ |
| Z, Calculated density | 4, 1.359 Mg/m³ |
| Absorption coefficient | 1.726 1/mm |
| F(000) | 1032 |
| Theta range for data collection | 3.020° to 67.647° |
| Limiting indices | −16 <= h <= 15; −14 <= k <= |
| | 14; −17 <= l <= 17 |
| Reflexion collected/unique | 35059/8540 [R(int) = 0.0565] |
| Completness to theta max | 99.1% |
| Refinement method | Full-matrix least-square on F² |
| Data/restraints/parameters | 8540/1/631 |
| Goodness of fit on F² | 0.917 |
| Final R indices [I > 2sigma(I)] | R1 = 0.0352; wR2 = 0.1058 |
| Final R indices [all data] | R1 = 0.0399; wR2 = 0.1131 |
| Absolute structure parameter | 0.014(6) |
| Largest diff peak and hole | 0.234 and −0.290 e/Å³ |

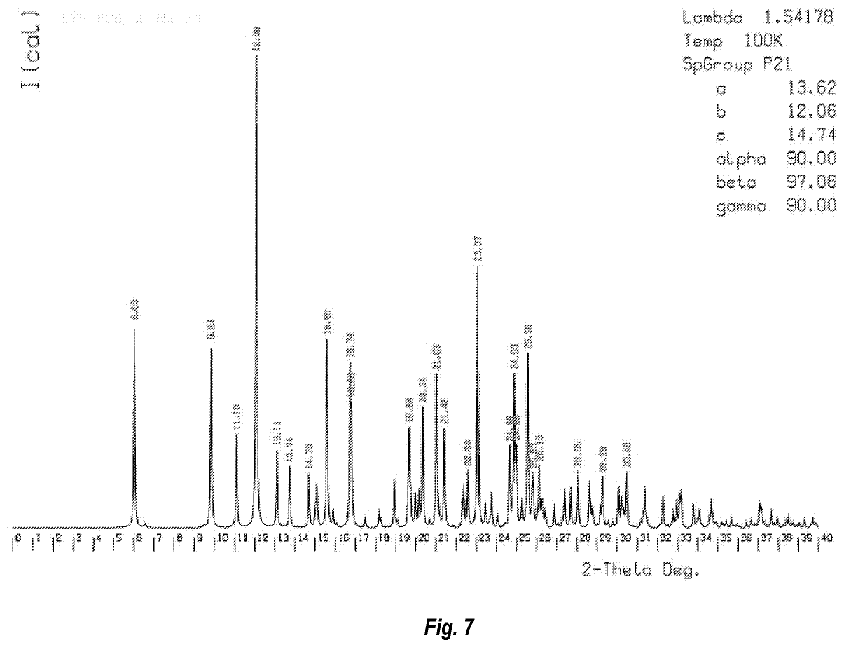

A simulated diffraction pattern (FIG. 7) was produced from the experimentally determined crystalline structure. An experimental powder diffraction pattern can be compared to this theoretical pattern to demonstrate the nature of the crystalline structure. Minor differences (if any) can be explained by asymmetric crystal morphology, particle size, or preferential orientations in the powder.

### 3.4 Crystal Data for Compound (1)·HCl Form 2

Ethyl acetate (AcOEt) and acetonitrile (ACN) in a ratio of 5/0.1 vol/vol was added to Compound 1·HCl Form 2 to form a slurry, which sat at RT for 3 days, then as a cold slurry for 4 days. More AcOEt/ACN mixture was added, followed by RT slurry for 1 day. Additional AcOEt/ACN mixture was added and then the solution underwent vacuum filtration.

Compound (1)·HCl Form 2 underwent XRPD analysis performed on a Bruker D2-Phaser diffractometer following those parameters:
Source CuKal, 1=1.5406Å.
Generator: 30 kV-10 mA.
Detector: Lynxeye SSD160 (1D mode)
Powder specimen holder
Rotating sample holder: 30 rpm
Angle range: 2° to 40° in 2-theta Bragg.
Step size: 0.03°
Step time: 0.5s by Step
PSD opening: 4.8°
Detector slit: 8 mm
X-Ray generator slit: 0.6 mm
Sample preparation: gently grinding
Space Group: P1
The unit cell parameters are given below:
   a(Å)=9.3589 b(Å)=12.3992 c(Å)=12.6660 α(°)=64.095 β(°)=70.641 γ(°)=74.644;

FIG. 8 shows an XRPD pattern of Compound (1)·HCl Form 2 obtained using Cu Kα radiation (wavelength: λ(Cu)=1.54178 Å).

Peaks identified in FIG. 8 include those set forth in Table 6:

TABLE 6

| Pos. [°2θ] | d-spacing [Å] | Height [cts] |
|---|---|---|
| 8.00 | 11.041 | 330.2 |
| 10.11 | 8.741 | 1439.0 |
| 11.98 | 7.380 | 327.1 |
| 13.33 | 6.635 | 260.4 |
| 14.40 | 6.148 | 325.4 |
| 14.92 | 5.931 | 856.5 |
| 15.66 | 5.653 | 311.6 |
| 16.05 | 5.517 | 398.0 |
| 16.72 | 5.298 | 213.3 |
| 17.28 | 5.128 | 356.3 |

### 3.5 Single Crystal Data and Structure Refinements of Compound (1)·HCl Form

A single crystal of Compound (1).HCl Form 2 (from crystals grown in a mixture of ethyl acetate (AcOEt)/acetonitrile (ACN)) was selected by observation under a binocular microscope and was mounted on the goniometric head of a Bruker APEX DUO Instrument equipped with a micro focused X-ray source (Bruker AXS (2015).APEX3 suite V2014.2-0 Madison, Wisconsin, U.S.A). Intensities were collected with the diffractometer at low temperature (T=112 K), with the use of a graphite monochromated Cu Kα radiation wavelength (λ=1.54178 Å). Systematic investigation of the diffraction nodes indicates that the crystal belongs to the triclinic system, with a primitive Bravais lattice. The unit cell parameters are given below:

a(Å)=9.39 b(Å)=12.31 c(Å)=12.40 α(°)=63.98 β(°)=73.90 γ(°)=69.66

In view of the number of atoms in the Compound 1·HCl Form 2 molecule and of the unit cell volume, it was concluded that this unit cell must contain two molecules having the formula C₂₆H₂₆ClN₅O₃, which is equivalent to a calculated density of 1.368. The number of reflections collected was 16793, of which 6829 were unique.

Based on the statistical distribution of the intensities, a non-centrosymmetric structure was deduced.

The structure was solved by direct methods using the XT dual-space module of SHELX; and was refined on F² by full least squares methods with SHELXTL, as set forth in Sheldrick, G. M. “A short history of SHELX,” Acta Crystallogr. Sect. A (2008) A64, 112-122. The molecular structure is well found, and all non-hydrogen atoms were refined with anisotropic displacement parameters; a riding model was used for hydrogen atoms. Final agreement values are R1=0.0273 (observed reflections) and wR2=0.0776 (all data) for 6829 reflections and 631 parameters, with a goodness of fit of 1.013.

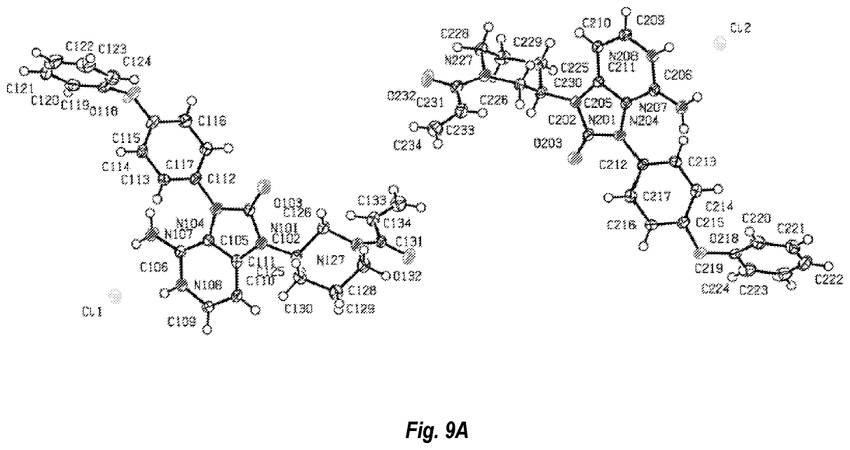

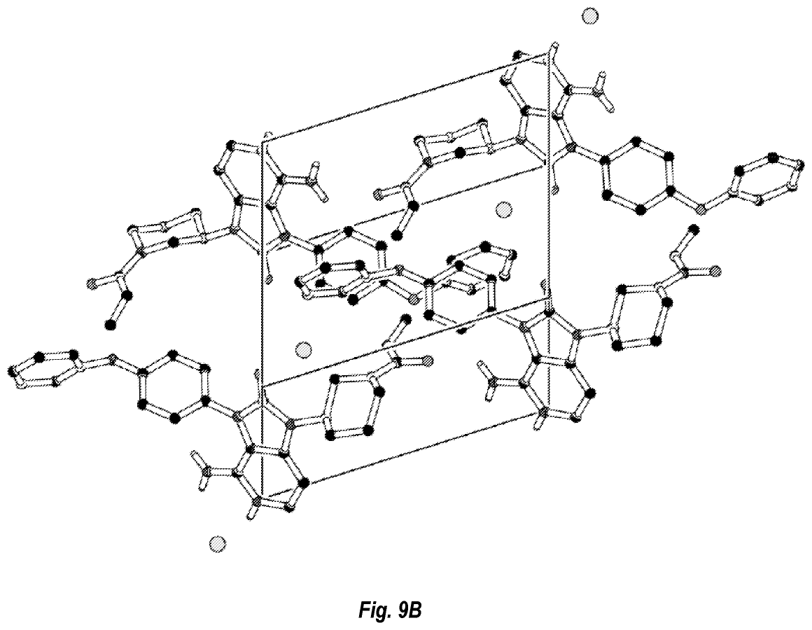

The compound crystallizes in the space group P 1 (N° 1), the asymmetric unit of the crystal is made up of two molecules of Compound (1) associated to their respective chlorine counter ion, and thus two formulae are present in the unit cell (see FIGS. 9A and 9B). The asymmetric cell therefore contains: [C₂₆H₂₆N₅O₃, Cl]. No additional molecule like organic solvent or water is found. Examination of the molecular structure confirms that all bond angles and lengths stand in the standard range values. There is no atomic disorder in the crystal. The salt bridge is established by the chlorine atom with the amino-imidazolopyridine nitrogen atom. Other non-covalent interactions are also present in the structure.

Crystal data, X-rays experimental parameters and structure refinements are given in Table 7.

TABLE 7
Crystal Data and structure refinements of Compound
1•HCl Form 2 by Single Crystal X-ray Diffraction

| Identification code | Compound 1•HCl Form 2 |
|---|---|
| Chemical formula | C₂₆ H₂₆ Cl N₅ O₃ |
| Molecular weight | 491.97 |
| Temperature | 112(2) |
| Wavelength | 1.54178 |
| Crystal system; space group | triclinic; P 1 |
| | a = 9.3921(19) Å; α = 63.99(3) ° |
| Unit cell dimensions | b = 12.310(3) Å; β = 73.90(3) ° |
| | c = 12.398(3) Å; γ = 69.66(3) ° |
| Volume | 1194.5(6) Å³ |
| Z, Calculated density | 2, 1.368 mg/m³ |
| Absorption coefficient | 1.737 1/mm |
| F(000) | 516 |
| Theta range for data collection | 4.012° to 65.244° |
| Limiting indices | −11 <= h <= 10; −14 <= k <= |
| | 14; −14 <= l <= 14 |
| Reflection collected/unique | 16793/6829 [R(int) = 0.0227] |
| Completeness to theta max | 99.0% |
| Refinement method | Full-matrix least-square on F² |
| Data/restraints/parameters | 6829/459/631 |
| Goodness of fit on F² | 1.013 |
| Final R indices [I > 2sigma(I)] | R1 = 0.0273; wR2 = 0.0769 |
| Final R indices [all data] | R1 = 0.0281; wR2 = 0.0776 |
| Absolute structure parameter | 0.041(5) |
| Largest diff peak and hole | 0.261 and −0.165 e/Å³ |

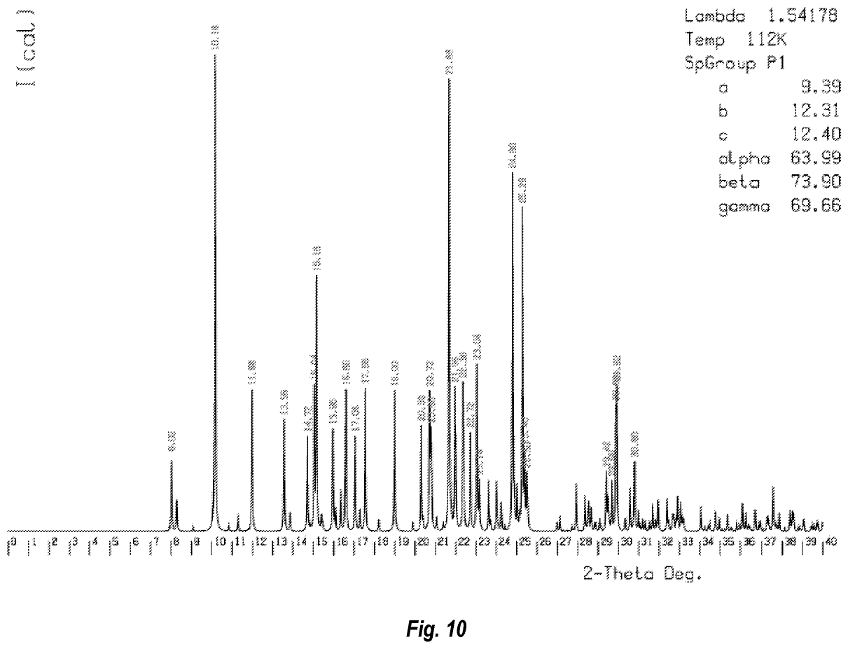

A simulated diffraction pattern (FIG. 10) was produced from the experimentally determined crystal structure of Compound 1·HCl Form 2. An experimental powder diffraction pattern can be compared to this theoretical pattern to demonstrate the nature of the crystalline structure. Minor differences (if any) can be explained by asymmetric crystal morphology, particle size, or preferential orientations in the powder.

EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

What is claimed is:

1. A substantially crystalline compound of Formula (1)

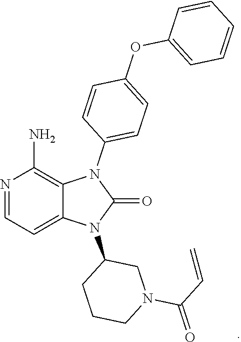 [SMILES: C=CC(=O)N1CCC[C@@H](n2c(=O)n(-c3ccc(Oc4ccccc4)cc3)c3c(N)nccc32)C1]

2. The substantially crystalline compound of claim 1, wherein the compound of Formula (1) is Form 1.

3. The substantially crystalline compound of claim 2, wherein the compound of Formula (1) Form 1 is characterized by an XRPD pattern substantially the same as FIG. 1.

4. The substantially crystalline compound of claim 3, wherein the compound of Formula (1) Form 1 is characterized by an XRPD pattern comprising one or more peaks chosen from peaks at about 7.66° 2θ, 7.86° 2θ, 10.03° 2θ, 10.51° 2θ, 10.97° 2θ, 11.99° 2θ, 13.19° 2θ, 13.59° 2θ and 13.96° 2θ.

5. The substantially crystalline compound of claim 1, wherein the compound of Formula (1) is Form 2.

6. The substantially crystalline compound of claim 5, wherein the compound of Formula (1) Form 2 is characterized by an XRPD pattern substantially the same as FIG. 2.

7. The substantially crystalline compound of claim 3, wherein the compound of Formula (1) Form 2 is characterized by an XRPD pattern comprising one or more peaks chosen from peaks at about 4.15° 2θ, 10.22° 2θ, 10.41° 2θ, 11.03° 2θ, 14.41° 2θ, 14.85° 2θ, 15.63° 2θ, 16.55° 2θ and 17.73° 2θ.

8. A substantially crystalline form of a compound of Formula (1)·HCl.

9. The substantially crystalline compound of claim 8, wherein the compound of Formula (1)·HCl is Form 1.

10. The substantially crystalline compound of claim 9, wherein the compound of Formula (1)·HCl Form 1 is characterized by an XRPD pattern substantially the same as FIG. 5.

11. The substantially crystalline compound of claim 9, wherein the compound of Formula (1)·HCl Form 1 is characterized by an XRPD pattern comprising one or more peaks chosen from peaks at about 6.309° 2θ, 9.480° 2θ, 10.933° 2θ, 12.261° 2θ, 12.647° 2θ, 14.482° 2θ, 14.918° 2θ, 16.253° 2θ and 16.425° 2θ.

12. The substantially crystalline compound of claim 8, wherein the compound of Formula (1)·HCl is Form 2.

13. The substantially crystalline compound of claim 12, wherein the compound of Formula (1)·HCl Form 2 is characterized by an XRPD pattern substantially the same as FIG. 8.

14. The substantially crystalline compound of claim 12, wherein the compound of Formula (1)·HCl Form 2 is characterized by an XRPD pattern comprising one or more peaks chosen from peaks at about 8.00° 2θ, 10.11° 2θ, 11.98° 2θ, 13.33° 2θ, 14.40° 2θ, 14.92° 2θ, 15.66° 2θ, 16.05° 2θ, 16.72° 2θ, and 17.28° 2θ.

15. The substantially crystalline compound of any one of the preceding claims, wherein the substantially crystalline compound is at least 85% crystalline.

16. The substantially crystalline compound of any one of the preceding claims, wherein the substantially crystalline compound is at least 90% crystalline.

17. The substantially crystalline compound of any one of the preceding claims, wherein the substantially crystalline compound is at least 95% crystalline.

18. The substantially crystalline compound of any one of the preceding claims, wherein the substantially crystalline compound is at least 97% crystalline.

19. The substantially crystalline compound of any one of the preceding claims, wherein the substantially crystalline compound is at least 99% crystalline.
